SEQUENCE LISTING (21 sequences)

SEQ ID NO: 1 - protein, 18 aa, Mus musculus
RGDSSSYKTWNLRAFGWY

SEQ ID NO: 2 - protein, 18 aa, Mus musculus
RGDNTIPDSQMSASSSYK

SEQ ID NO: 3 - protein, 18 aa, Mus musculus
RGDSSSYKTWNLRAFGWY

SEQ ID NO: 4 - protein, 18 aa, Mus musculus
RGDAFGWYPHLGRLDNQG

SEQ ID NO: 5 - protein, 18 aa, Mus musculus
RGDLDNQGKINAWTAQSN

SEQ ID NO: 6 - protein, 18 aa, Mus musculus
RGDTAQSNSAKEWLQVDL

SEQ ID NO: 7 - protein, 18 aa, Mus musculus
RGDLQVDLGTQRQVTGII

SEQ ID NO: 8 - protein, 18 aa, Mus musculus
RGDVTGIITQGARDFGHI

SEQ ID NO: 9 - protein, 18 aa, Mus musculus
RGDDFGHIQYVASYKVAH

SEQ ID NO: 10 - protein, 18 aa, Mus musculus
RGDYKVAHSDDGVQWTVY

SEQ ID NO: 11 - protein, 18 aa, Mus musculus
RGDQWTVYEEQGSSKVFQ

SEQ ID NO: 12 - protein, 18 aa, Mus musculus
RGDSKVFQGNLDNNSHKK

SEQ ID NO: 13 - protein, 18 aa, Mus musculus
RGDNSHKKNIFEKPFMAR

SEQ ID NO: 14 - protein, 18 aa, Mus musculus
RGDPFMARYVRVLPVSWH

SEQ ID NO: 15 - protein, 20 aa, Mus musculus
RGDPVSWHNRITLRLELLGC

SEQ ID NO: 16 - protein, 18 aa, Homo sapiens
RGDSSSYKTWGLHLFSWN

SEQ ID NO: 17 - protein, 463 aa, Mus musculus
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 18 - protein, 30 aa, Mus musculus
CDSSLCLNGGTCLTGQDNDIYCLCPEGFTG

SEQ ID NO: 19 - protein, 39 aa, Mus musculus
CSPNPCYNDAKCLVTLDTQRGDIFTEYICQCPVGYSGIH

SEQ ID NO: 20 - protein, 56 aa, Mus musculus
CSTQLGMEGGAIADSQISASSVYMGFMGLQRWGPELARLYRTGIVNAWTASNYDSK

SEQ ID NO: 21 - protein, 157 aa, Mus musculus
GCSCPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

(19) United States
(12) Patent Application Publication
(10) Pub. No.: US 2024/0197828 A1
(43) Pub. Date: Jun. 20, 2024

(54) Chimeric molecule to treat sepsis and other inflammatory conditions

(71) Applicant: THE FEINSTEIN INSTITUTES FOR MEDICAL RESEARCH, Manhasset, NY (US)

(72) Inventors: Ping Wang, Roslyn, NY (US); Colleen Nofi, Huntington, NY (US); Monowar Aziz, New Hyde Park, NY (US); Max Brenner, Briarwood, NY (US)

(73) Assignee: THE FEINSTEIN INSTITUTES FOR MEDICAL RESEARCH, Manhasset, NY (US)

(21) Appl. No.: 18/538,023

(22) Filed: Dec. 13, 2023

Related U.S. Application Data
(60) Provisional application No. 63/433,789, filed on Dec. 20, 2022.
(60) Provisional application No. 63/540,688, filed on Sep. 27, 2023.

(51) Int. Cl. A61K 38/18; A61P 29/00; A61P 31/00

(52) U.S. Cl. CPC A61K 38/1808; A61P 29/00; A61P 31/00

(57) ABSTRACT

Milk fat globule-epidermal growth factor—factor VIII (MFG-E8) derived oligopeptides and pharmaceutical compositions containing the oligopeptides are provided for treating sepsis and inflammatory conditions.

### CROSS-REFERENCE TO RELATED APPLICATIONS

This application claims the benefit of U.S. Provisional Patent Application No. 63/433,789, filed on Dec. 20, 2022, and U.S. Provisional Patent Application No. 63/540,688, filed on Sep. 27, 2023, the contents of both of which are herein incorporated by reference in their entirety into the present application.

### STATEMENT OF GOVERNMENT SUPPORT

This invention was made with government support under grant numbers GM118337, HL076179, AA028947, AI133655, AI170018, and GM129633 awarded by the National Institutes of Health. The government has certain rights in the invention.

### INCORPORATION BY REFERENCE OF SEQUENCE LISTING XML

The Sequence Listing entitled “818.xml”, created on Nov. 27, 2023, 25 KB, submitted electronically using the U.S. Patent Center is incorporated by reference as the Sequence Listing XML for the subject application.

### BACKGROUND

Throughout this application, various publications are referred to in parentheses. Full citations for these may be found at the end of the specification immediately preceding the claims. The disclosures of these publications are hereby incorporated by reference in their entireties into the subject application to more fully describe the art to which the subject application pertains. The discussion of these publications herein is intended merely to summarize the assertions made by Applicant and no admission is made that any publication constitutes prior art.

Sepsis refers to life-threatening organ dysfunction caused by dysregulated host response to an infection (1). It inflicts over 50 million patients per year, resulting in approximately 11 million deaths worldwide (2). Unfortunately, therapeutic options in sepsis are still limited to source control and supportive care, including antibiotics, resuscitation, and hemodynamic support. Despite increasing funds with over $476 million in funding dedicated by the National Institutes of Health (NIH) to sepsis research over the past decade, advancements have improved our understanding of pathophysiologic mechanisms without translating to practice-changing therapies for afflicted patients (3). Thus, there remains an urgent need for uncovering critical inflammatory pathways and developing better-targeted therapies to improve patient outcomes.

A recently discovered and important sepsis mediator is extracellular cold-inducible RNA-binding protein (eCIRP) (4). Under physiologic conditions, intracellular CIRP is a 17-kDa chaperone for RNA molecules (4). In response to inflammatory and noxious stimuli, CIRP escapes into the extracellular space, thereby functioning as a pro-inflammatory damage-associated molecular pattern (DAMP) or chromatin-associated molecular pattern (CAMP) (5,6). As a potent CAMP, eCIRP binds its receptors, Toll-like receptor 4 (TLR4) or triggering receptor expressed on myeloid cells-1 (TREM-1), and contributes to propagation of the inflammatory cascade, known as the “cytokine storm” during an early stage of sepsis (7). Clinically, high levels of circulating eCIRP have been correlated with worse sepsis severity and mortality (4,8). In pre-clinical models of sepsis, eCIRP contributed to dysregulated inflammatory responses and associated organ injury (7); whereas pharmacological suppression of eCIRP with neutralizing antibodies or peptide antagonists conferred significant protection (9,10).

In contrast, milk fat globule-epidermal growth factor—factor VIII, MFG-E8, is a 64-kDa glycoprotein that occupies an anti-inflammatory role in sepsis (11). MFG-E8 is ubiquitously expressed by professional and non-professional phagocytes, and can be secreted extracellularly to serve as an opsonin for apoptotic cells (11,12). Structurally, MFG-E8 is comprised of EGF-like domains at the N-terminal and tandem F5/8-type C domains and the C-terminal, otherwise referred to as discoidin-1 (DD-1) and discoidin-2 (DD-2) domains (11). Through recognition of “eat me” signal phosphatidyl-serine, MFG-E8 recognizes apoptotic cells at its discoidin domains, and links them to phagocytes by interacting with the auß3-integrin receptor through preserved RGD-motif (within EGF-like domains), thereby promoting phagocytosis of apoptotic cells to attenuate inflammation (11-13). The important role of apoptotic cell scavenging is further exemplified in autoimmune diseases, such as MFG-E8 deficient elderly animals autonomously developed systemic lupus erythematosus (SLE) (14). In animal models of experimental sepsis, MFG-E8 expression is dramatically reduced, whereas its supplementation conferred significant protection against sepsis-induced inflammation and organ injury (13,15-17).

Given the anti-parallel increase of eCIRP (4, 8) and decrease of MFG-E8 levels in experimental and clinical sepsis (11), these two proteins may interact with each other to tilt the immune balance in sepsis. The present invention provides a novel small therapeutic MFG-E8-derived oligopeptide as an opsonic agent to clear eCIRP and prevent eCIRP-induced inflammation and acute lung injury (ALI) in sepsis and other diseases.

### SUMMARY OF THE INVENTION

The invention provides milk fat globule-epidermal growth factor—factor VIII (MFG-E8) derived oligopeptides and pharmaceutical compositions containing the oligopeptides for treating sepsis and inflammatory conditions. In one embodiment, the oligopeptide has the amino acid sequence RGDSSSYKTWNLRAFGWY (SEQ ID NO. 1) or RGDSSSYKTWGLHLFSWN (SEQ ID NO. 16).

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Identification of MOP peptides. Fragmentation of DD-2 of murine MFG-E8 into 15 segments, subsequently tagged with RGD to create 15 test peptides.

FIG. 2. Effects of MOP peptides on TNFα levels. RAW264.7 macrophages were cultured and treated with either volume-equivalent PBS, recombinant murine (rm) CIRP (eCIRP; 1 μg/mL), or rmCIRP with each respective peptide (10 μg/mL). After 4 hours, cell supernatants were assessed for TNFα levels by ELISA. Experiments were performed 3 times, and all data were used for analysis. Data are expressed as mean±SEM and compared by one-way ANOVA and SNK test. *p<0.05 vs. PBS, #p<0.05 vs. eCIRP.

FIG. 3. Effects of MOP peptides MOP-3, MOP-8 and MOP-14 on eCIRP. Flow cytometry experiment whereby RAW264.7 macrophages were treated for 1 hour according to respective group: PBS (volume-equivalent), FITC-rmCIRP (1 μg/mL), or a mixture of FITC-rmCIRP (1 μg/mL) with each respective peptide (10 μg/mL) after co-incubating for 30 minutes. Intracellular FITC-rmCIRP was analyzed by flow cytometry. Median fluorescence intensity (MFI) reported as percentage relative to PBS-treated RAW264.7 cells. Data from two independent experiments are expressed as mean±SEM and compared by one-way ANOVA and SNK test. *p<0.05 vs. PBS, #p<0.05 vs. eCIRP.

FIG. 4. eCIRP-induced inflammation is attenuated by MOP3. RAW264.7 cells were treated according to respective groups: PBS (volume equivalent), rmCIRP (1 μg/mL), or rmCIRP with MOP3 at increasing doses. After 4 hours, cell supernatants were analyzed for TNFα levels by ELISA. Experiments were performed 3 times, and all data were used for analysis. Data are expressed as mean #SEM and compared by one-way ANOVA and SNK test. *p<0.05 vs. PBS, #p<0.05 vs. eCIRP.

FIG. 5A-5B. eCIRP-induced inflammation is attenuated by MOP3. WT mice were treated according to respective groups: PBS (volume equivalent), rmCIRP (5 mg/kg), or MOP3 (10 μg/kg) co-incubated with rmCIRP (5 mg/kg) for 30 mins prior to r.o. injection. After 4 hours, serum was measured for IL-6 (A) and TNFα (B) levels by ELISA. All experiments were performed 3 times, and all data were used for analysis (N=7-10). Data are expressed as mean±SEM and compared by one-way ANOVA and SNK test. *p<0.05 vs. PBS, #p<0.05 vs. eCIRP.

FIG. 6A-6M: MOP3 ameliorates inflammation, lung injury, and improves survival in sepsis. WT mice subjected to CLP-induced sepsis with MOP3 treatment (10 μg/kg) or vehicle (volume-equivalent). After 20 hours, blood and lung tissue collected for respective analyses. (A-C) Serum was analyzed for systemic eCIRP, IL-6, and TNFα levels by ELISA. (D-F) Serum was analyzed for ALT, AST, and LDH by calorimetric assays. (G-I) Lung tissue was frozen and mRNA expression of IL-6, TNFα, and IL-1β was measured by PCR and MPO by calorimetric assay. (J) Lung tissue was frozen and analyzed for MPO levels by calorimetric assay. (K) Lungs were sectioned, imaged for histologic injury by H&E staining, and performed lung injury scoring. (L) Sectioned lungs were stained for TUNEL positive cells and cell counts were quantified. All experiments were performed 3 times, and all data were used for analysis (N=6-18). Data are expressed as mean±SEM and compared by one-way ANOVA and SNK test. *p<0.05 vs. sham, #p<0.05 vs. CLP vehicle. (M) WT mice subjected to CLP-induced sepsis with MOP3 treatment (10 μg/kg) or vehicle (volume equivalent) and monitored for 10 days for humane endpoints, and differences in survival were determined (N=17 per group). *p<0.05 vs. CLP vehicle.

FIG. 7A-7C. MOP3 reduces tissue injury in CLP-induced sepsis. (A) aspartate aminotransferase (AST), (B) alanine transaminase (ALT), (C) lactate dehydrogenase (LDH).

### DETAILED DESCRIPTION OF THE INVENTION

The invention provides a method of treating a subject with sepsis or with an inflammatory condition comprising administering to the subject a therapeutic amount of a peptide selected from the group consisting of one or more of MOP3, MOP3H, MOP8 and MOP14, or a nucleic acid encoding the peptide, wherein

MOP3 has the amino acid sequence
(SEQ ID NO. 1)
RGDSSSYKTWNLRAFGWY,
MOP3H has the amino acid sequence
(SEQ ID NO. 16)
RGDSSSYKTWGLHLFSWN,
MOP8 has the amino acid sequence
(SEQ ID NO: 8)
RGDVTGIITQGARDFGHI,
and
MOP14 has the amino acid sequence
(SEQ ID NO: 14)
RGDPFMARYVRVLPVSWH.

In one embodiment, the method consists of administering one or both of MOP3 and MOP3H to the subject.

In one embodiment, treatment of the subject with the peptide reduces tissue injury in the subject compared to tissue injury that would occur in the absence of treatment with the peptide. In one embodiment, lung injury is reduced by treatment of the subject with the peptide.

In one embodiment, the inflammatory condition is one or more of appendicitis, peptic, gastric or duodenal ulcers, peritonitis, pancreatitis, ulcerative colitis, pseudomembranous, acute or ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, necrotizing enterocolitis, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, bums, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, adult respiratory distress syndrome, Alzheimer's disease, meningitis, encephalitis, multiple sclerosis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcet's syndrome, allograft rejection, graft-versus-host disease, ankylosing spondylitis, Type I diabetes, ankylosing spondylitis, Berger's disease, reactive arthritis (Reiter's syndrome) or Hodgkin's disease. In more preferred embodiments, the condition is appendicitis, peptic, gastric or duodenal ulcers, peritonitis, pancreatitis, ulcerative colitis, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, necrotizing enterocolitis, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection, graft-versus-host disease and bacterial infection.

The invention further provides a pharmaceutical composition comprising a pharmaceutically acceptable carrier and a peptide selected from the group consisting of one or more of MOP1, MOP2, MOP3, MOP3H, MOP4, MOP5, MOP6, MOP7, MOP8, MOP9, MOP10, MOP11, MOP12, MOP13, MOP14 and MOP15. In one embodiment, the peptide is one or both of MOP3 and MOP3H. As used herein, a “pharmaceutically acceptable carrier” is (i) compatible with the other ingredients of the composition without rendering the composition unsuitable for its intended purpose, and (ii) suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include any of the standard pharmaceutical carriers such as phosphate-buffered saline solutions, water, and emulsions such as oil/water emulsions and microemulsions.

### EXPERIMENTAL DETAILS

### Example 1

### Methods

Computational modeling was used to determine the likely domain of MFG-E8's binding to eCIRP. The known amino acid (AA) sequence of this domain was used to develop and test potential therapeutic peptides. An MFG-E8 derived oligopeptide, MOP3, was developed based on 15 AA from MFG-E8 discoidin 2 domain (mimicking MFG-E8's C-terminal) and tagged with the 3 AA, RGD, (mimicking MFG-E8's binding capacity for the integrin receptor at the N-terminal). Binding of MOP3 to CIRP was quantified using Surface Plasmon Resonance (SPR) analysis. Macrophages (RAW264.7 cells) were treated with FITC-recombinant mouse (rm)-CIRP or co-incubated MOP3 and FITC-rmCIRP with and without a. Br-integrin Ab. After 1 h, cells were fixed and analyzed for intracellular FITC-rmCIRP by flow cytometry. Macrophages were stimulated with rmCIRP or co-incubated mixture of MOP3 and rmCIRP. After 4 h, cell culture supernatants were analyzed for inflammatory cytokine, TNFα.

### Results

MOP3 binds rmCIRP theoretically with a binding energy of −6.6 Kcal/mol. This interaction was experimentally quantified and exhibited strong binding affinity with a Kp of 1.3×10⁻⁸ M. RAW264.7 macrophages treated with MOP3-rmCIRP co-incubated mixture had significantly increased intracellular rmCIRP uptake (similar to the effect of MFG-E8) and significantly decreased TNFα levels in cell culture supernatants than cells treated with rmCIRP alone. Blocking the avBa-integrin receptor significantly reduced MFG-E8-mediated uptake of rmCIRP.

### Conclusion

The novel small peptide therapeutic, MOP3, can replace MFG-E8's anti-inflammatory function by clearing eCIRP from circulation through an integrin-mediated pathway and reducing inflammation. Given MFG-E8's demonstrated beneficial effect in a multitude of disease states, MOP3 may similarly protect against eCIRP-induced cell and tissue injury during acute inflammatory conditions.

### SEQUENCE INFORMATION

| Full length mouse MFG-E8 Sequence (NP_032620; 463 aa) | |
|---|---|
| (SEQ ID NO: 17) | |
| 1 | mqvsrvlaal cgmllcasgl faasgdfcds slclnggtcl tgqdndiycl cpegftglvc |
| 61 | netergpcsp npcyndakcl vtldtqrgdi fteyicqcpv gysgihcete tnyynldgey |
| 121 | mfttavpnta vptpaptpdl snnlasrcst qlgmeggaia dsqisassvy mgfmglqrwg |
| 181 | pelarlyrtg ivnawtasny dskpwiqvnl lrkmrvsgvm tqgasragra eylktfkvay |
| 241 | sldgrkfefi qdesggdkef lgnldnnslk vnmfnptlea qyiklypvsc hrgctlrfel |
| 301 | lgcelhgcse plglknntip dsqmsasssy ktwnlrafgw yphlgrldnq gkinawtaqs |
| 361 | nsakewlqvd lgtqrqvtgi itqgardfgh iqyvasykva hsddgvqwtv yeeqgsskvf |
| 421 | qgnldnnshk knifekpfma ryvrvlpvsw hnritlrlel lgc |

### The 4 Domains:

1. EGF1 (28..57):
(SEQ ID NO: 18)
28-cds slclnggtcl tgqdndiycl cpegftg-57
2. EGF2 (68..106):
(SEQ ID NO: 19)
68-csp npcyndakcl vtldtqrgdi fteyicqcpv gysgih-106
3. Discoidin domain 1 (148..303):
(SEQ ID NO: 20)
148-cst qlgmeggaia dsqisassvy mgfmglqrwg pelarlyrtg ivnawtasny dsk-303
4. Discoidin domain 2 (307..463):
(SEQ ID NO: 21)
307-gcsc plglknntip dsqmsasssy ktwnlrafgw yphlgrldnq gkinawtaqs
nsakewlqvd lgtqrqvtgi itqgardfgh iqyvasykva hsddgvqwtv yeeqgsskvf
qgnldnnshk knifekpfma ryvrvlpvsw hnritlrlel lgc-463

All peptide sequences from mouse discoidin domain 2:

Peptide#1
(SEQ ID NO: 1)
RGDgcscplglknntipd (MOP1)
Peptide#2
(SEQ ID NO: 2)
RGDntipdsqmsasssyk (MOP2)
Peptide#3
(SEQ ID NO: 3)
RGDsssyktwnlrafgwy (MOP3)
Peptide#4
(SEQ ID NO: 4)
RGDafgwyphlgrldnqg (MOP4)
Peptide#5
(SEQ ID NO: 5)
RGDldnqgkinawtaqsn (MOP5)
Peptide#6
(SEQ ID NO: 6)
RGDtaqsnsakewlqvdl (MOP6)
Peptide#7
(SEQ ID NO: 7)
RGDlqvdlgtqrqvtgii (MOP7)
Peptide#8
(SEQ ID NO: 8)
RGDvtgiitqgardfghi (MOP8)
Peptide#9
(SEQ ID NO: 9)
RGDdfghiqyvasykvah (MOP9)
Peptide#10
(SEQ ID NO: 10)
RGDykvahsddgvqwtvy (MOP10)
Peptide#11
(SEQ ID NO: 11)
RGDqwtvyeeqgsskvfq (MOP11)
Peptide#12
(SEQ ID NO: 12)
RGDskvfqgnldnnshkk (MOP12)
Peptide#13
(SEQ ID NO: 13)
RGDnshkknifekpfmar (MOP13)
Peptide#14
(SEQ ID NO: 14)
RGDpfmaryvrvlpvswh (MOP14)
Peptide#15
(SEQ ID NO: 15)
RGDpvswhnritlrlellgc (MOP15)
“MOP3” is Peptide 3-18 AA Sequence:
(SEQ ID NO: 3)
RGD-SSSYKTWNLRAFGWY

If MOP3 18AA Sequence were to be based on human discoidin 2 domain:

(SEQ ID NO: 16)
RGDSSSYKTWGLHLFSWN (MOP3H)

### Example 2

### Materials and Methods

### Study Design

Adult (8-12 weeks) male, C57BL/6 wild-type (WT) mice (20-30 g) were purchased from Charles River Laboratories. MFG-E8 knockout mice (MFG-E8^{-/-}) were originally obtained from Dr. Shigekazu Nagata, Osaka University of Japan, and bred following our Institutional Animal Care and Use Committee (IACUC) policies. Age-matched, healthy mice were used in all experiments. Animals were randomly assigned to sham, vehicle, or treatment groups. Concerted effort was made to ensure that a limited number of animals were used in each experiment. All experiments were previously approved by IACUC of the Feinstein Institutes for Medical Research (Manhasset, NY) and were performed in accordance with the guidance for the use of experimental animals by the National Institutes of Health (Bethesda, MD). For the design of in vivo models, sample size calculation was driven by variability, effect size, significance level and power as determined through performance of early feasibility and optimization pilot studies.

### Recombinant Proteins and Peptides

For Biacore experiments, recombinant murine (rm) CIRP was purchased from Cubasio Technology LLC (Cat #. CSB-EP613483He0), rmMFG-E8 was purchased from R&D Systems (Cat #2767-MF-050), and auß3-integrin was purchased from R&D Systems (Cat #7889-AV-050). For in vitro studies, recombinant human (rh)- and rmMFG-E8 and rmCIRP was produced by our lab as previously described (4). MFG-E8-derived oligopeptide 3, MOP3, was developed based on theoretical analyses demonstrating strongest binding between the eCIRP and DD-2 of MFG-E8. The known AA sequence of DD-2 (157 amino acids long) of murine MFG-E8 was split into ˜15 AA long sequences with a 5 AA long overlap. This portion of the peptide was designed to bind to eCIRP. The 15 AA sequences were then all tagged with three AA long, Arg-Gly-Asp (RGD), designed to bind to the integrin receptor. This process resulted in the production of 15 peptides (synthesized by GenScript USA Inc, Piscataway NJ). All peptides were screened in vitro for the ability to attenuate eCIRP-induced TNFα production. Three peptides of interest underwent further testing by flow cytometry and Biacore analyses to confirm their ability to link eCIRP to the αᵥβ₃-integrin receptor, resulting in greater clearance of eCIRP. These experiments yielded one peptide of interest, MFG-E8-derived oligopeptide 3, named MOP3.

### SPR BIAcore

To examine the direct interaction between rmCIRP and rmMFG-E8, surface plasmon resonance (SPR), OpenSPR (Nicoya), was performed between rmCIRP and rmMFG-E8. rmCIRP was immobilized on carboxyl sensor and rmMFG-E8 was injected as an analyte in concentrations of 15.7 nM to 250 nM. Binding reactions were performed in DPBS, with Calcium and Magnesium, 0.05% P20, 1% BSA, and pH 7.4. In parallel experiments, binding of MOP3 to rmCIRP (analyte) was performed with concentrations of 0.5 μm to 2 μM, and binding of MOP3 to αᵥβ₃-integrin (analyte) was performed in concentrations of 31.25 nM to 125 nM. All binding reactions were performed in PBS 0.05% P20, pH7.4. Briefly, the carboxyl sensor was first cleaned by injection 10 mM HCL 150 μl, followed by injection of 150 μl of the mixture of 1 aliquot of N-ethyl-N′-[3-diethylaminopropyl]-carbodiimide (EDC) and 1 aliquot of N-hydroxysuccinimide (NHS) to activate the sensor surface. An aliquot of 200 μl of 50 μg/ml of the ligand diluted in 10 mM sodium acetate (pH 5.5) was injected at 20 μl/minutes into flow cell-channel-2 of the sensor for immobilization. Next, 150 μl of 1 M ethylenediamine (pH 8.5) was injected to deactivate the remaining active sites on channel 1&2. The flow cell-1 was used as a control to evaluate nonspecific binding. The binding analyses were performed at a flow rate of 40 μl per minute at 20° C. 1M NaCl was used for regeneration. To evaluate binding, the analyte was injected into flow cell-1 and flow cell-2 and increasing concentrations and real-time interaction data were analyzed by TraceDrawer (Nicoya). The signals from the control channel (flow cell-1) were subtracted from the channel coated with the ligand (flow cell-2) for all samples. Data were globally fitted for 1:1 binding.

### Cell Line and Culture Condition

Mouse macrophage cell line, RAW264.7 cells were obtained from American Type Culture Collection (ATCC) and cultured in DMEM media with 10% FBS, 1% penicillin and streptomycin, and 1% glutamine. Cells were kept in 37° C. incubator under humidified conditions containing 5% CO₂.

### Isolation of Peritoneal Macrophages

Murine peritoneal cavity (PerC) macrophages were isolated from adult, WT mice and MFG-E8“ ” mice. Briefly, mice were euthanized using CO₂ asphyxiation. Peritoneal fluid and cells were isolated using peritoneal lavage by washing ice-cold Ca⁺² and Mg⁺² free Hank's Balanced Salt Solution (HBSS) with 2% fetal bovine serum (FBS, Solon, Ohio). Total PerC macrophages were isolated by centrifugation at 400 g for 10 minutes at 4° C. After centrifugation, the cell pellet was suspended in culture media containing RPMI 1640 (Invitrogen) supplemented with 25 mM HEPES, 2 mM glutamine, 10% FBS, penicillin (100 IU/mL) and streptomycin (100 IU/mL). PerC macrophages were allowed to adhere in 6-well plates for 4 hours in humidified incubator at 37ºC. Non-adherent cells were removed by washing with pre-warmed culture media. Adhered PerC macrophages were then mechanically detached from the plate and counted. Approximately 3×10⁴ PerC macrophages were added to a 96-well flat-bottom cell culture plate and incubated overnight prior to use for experiments.

### In Vitro Experiments

RAW264.7 cells were cultured, seeded in 96-well plate (approximately 3×10⁴ cells/well), and treated in Optimem media with either PBS, rmMFG-E8 (500 ng/ml), rmCIRP (1 μg/mL), or mixture of rmMFG-E8 and rmCIRP (allowed to co-incubate for 30 minutes) given as simultaneous treatment. Cells were treated for 4 hours and collected for use for several experiments. In similar fashion, murine PerC macrophages were isolated as described from either WT or MFG-E8^{-/-} mice and treated with either PBS (control) or rmCIRP (1 μg/mL). For in vitro peptide testing, cells were cultured in parallel fashion, and treated with respective doses of MOP3.

### Protein-Fluorophore Labeling

rmCIRP was labeled using a FITC Conjugation Kit (Abcam, ab188285), and rmMFG-E8 was labeled using Alexa Fluor Conjugation Kit (Abcam, ab269821), both according to respective manufacturer's instructions.

### Antibodies, Fluorescent Probes and Dyes

Fluorophore labeled proteins (FITC-rmCIRP and AlexaFluor568-rmMFG-E8) were utilized in respective experiments as described. Tetramethylrhodamine albumin from bovine serum (TRITC-BSA, Invitrogen, A23016) was used as a marker for endocytosis and intracellular protein processing. Lysosome visualization was achieved through primary antibody staining, anti-lysosomal associated membrane protein 1 (LAMP1) antibody (Abcam, ab208943) with secondary probes in respective experiments (donkey anti-rabbit IgG antibody Alexa Fluor 594 (Invitrogen, A-21207), or goat anti-rabbit IgG Alexa Fluor 647 (Invitrogen, A-32728)). For live cell imaging, lysosomal visualization was achieved utilizing LysoTracker Deep Red (Invitrogen, L12492). Additional macrophage membrane stains (MemBrite Fix 405/430 Cell Surface Staining kit, (Biotium 30092)), cytosol stains (Celltrace violet (Invitrogen, C34557)) and nucleic acid stains (NucBlue Live Cell Stain, (Invitrogen, R37605), or Syto Deep Red Fluorescent Nucleic acid stain (Invitrogen, S34900)) were also utilized, all according to manufacturers' instructions.

### Immunofluorescence Imaging and Assays

In order to visualize cellular uptake, we initially utilized TRITC-BSA as an internal probe, as BSA is taken up into cells primarily by endocytosis and retained intracellularly after fixation (27, 28). For further visualization of intracellular localization, we utilized LAMP1 primary antibody (as it is distributed among endolysosomal organelles and serves as a marker for lysosomal compartments) (29), and utilized lysotracker in live cell experiments (as it allows for imaging of dynamics of lysosome vesicle movements) (30). RAW264.7 cells were treated in various conditions with rmCIRP, rmMFG-E8, and inhibitory antibody against auß3-integrin (Bioss antibodies, bs-1310R) for 4 hours. Cells were fixed with 4% paraformaldehyde (PFA), washed with 1×PBS, permeabilized using 0.3% Triton x100, and blocked using 5% BSA 100 mM Glycine in PBS prior to primary antibody treatment. After incubation with secondary antibodies, cells were washed and mounted with ProLong Gold antifade reagent (Invitrogen). For live cell imaging, cells remained in treatment conditions and were imaged under CO₂. Staining procedures were performed according to the manufacturers' instructions utilizing respective fluorescent dyes and probes. For all imaging, confocal microscopy images were obtained using a Zeiss LSM880 confocal microscope equipped with a 63× objective. Images were quantified and analyzed using Image J, FIJI.(31) Flow cytometry.

To detect changes in internalized eCIRP in the presence of MFG-E8, RAW264.7 cells were seeded in 12-well plates (approximately 3×10⁵ cells/well) and were treated with FITC-eCIRP. Treatment groups consisted of FITC-eCIRP, FITC-eCIRP with MFG-E8, FITC-eCIRP with MFG-E8 and αᵥβ₃-integrin antibody (0.5 μg/mL), or FITC-eCIRP with MFG-E8 and IgG isotype (0.5 μg/mL). Similar experiments were conducted with MOP3 instead of MFG-E8 in parallel groups. After 1 hour, cells were washed with PBS, mechanically suspended, and fixed in 4% paraformaldehyde. Unstained cells were used as a negative control to establish the flow cytometer (BD Biosciences). Acquisition was performed on 10,000 events using a BD LSRFortessa flow cytometer (BD Biosciences), and data were analyzed with FlowJo software (Tree Star).

In Vivo Administration of rmCIRP, rmMFG-E8, and MOP3

Mice were treated through retro-orbital (r.o.) injection according to respective treatment group (i.e. PBS, rmCIRP, rmMFG-E8, rmCIRP and rmMFG-E8 co-incubated for 30 minutes prior to co-treatment, or rmCIRP and MOP3 similarly incubated for 30 minutes and given as co-treatment). Injections were administered using a 29G×½″ U-100 insulin syringe (Terumo Medical Corporation). rmCIRP was given at a dose of 5 mg/kg body weight (BW), rmMFG-E8 was given at a dose of 40 μg/kg BW, MOP3 was given at a dose of 10 mg/kg BW, and vehicle mice were administered the volume-equivalent solution. 4 hours after r.o. injection, mice were euthanized with CO₂ asphyxiation and blood was collected, centrifuged at 4,000 RPM for 10 minutes, and serum separated for future analyses. Lung tissue was collected and either flash frozen or stored in 10% formalin for respective analyses.

### Murine Model of Polymicrobial Sepsis

Sepsis was induced in mice by cecal ligation and puncture (CLP) as previously described (4). Briefly, mice were anesthetized with inhaled isoflurane to achieve adequate anesthesia. After placement in the supine position, the abdomen was shaved and disinfected. A 2-cm midline laparotomy was created. The cecum was located and eviscerated from the abdomen and then ligated with a 4-0 silk suture approximately 1 cm proximal from the terminal cecal extremity. For short-term experiments using this polymicrobial sepsis model, the cecum was punctured twice (through and through) with a 22G needle. Careful extraction of a consistent, small amount of cecal content was extruded, and the cecum along with cecal content was returned to the peritoneal cavity. For long-term polymicrobial sepsis models (utilized in 10-day experiments), the length of cecum included in ligation and amount of cecal content extruded was slightly reduced, the cecum was punctured only once with a 22G needle, and mice were additionally treated with Imipenem/Cilastatin to achieve an LD50 model. All comparative surgeries were performed on the same day to reduce severity-variability. All short-term experiments were repeated at least three-times to ensure reproducibility of relative markers of inflammation and injury.

For respective experiments, either WT or MFG-E8 knockout mice were subjected to CLP. For treatment experiments, mice were randomly assigned to either vehicle or treatment groups. MOP3 (10 mg/kg) was given via retroorbital (r.o.) injection at time of surgery, immediately following abdominal closure. Vehicle groups received an equivalent volume of normal saline via r.o. injection. For resuscitation, mice additionally received subcutaneous injection of 500 μL of normal saline immediately after abdominal closure.

### Enzyme-Linked Immunosorbent Assay (ELISA)

The mouse macrophage cell culture supernatants were analyzed for cytokine levels using ELISA kits (BD Biosciences, CA). Macrophages were treated with either PBS (control), rmMFG-E8, rmCIRP, co-incubated mixture of rmMFG-E8 and rmCIRP, or co-incubated mixture of MOP3 and rmCIRP for 4 hours prior to cell supernatant collection. TNF-α and IL-6 levels were measured in cell supernatants by ELISA in accordance with respective manufacturers' instructions (BD Biosciences, CA). Blood was collected, centrifuged at 4,000 RPM for 10 minutes, and sera was separated and analyzed for cytokine levels using ELISA kits (BD Biosciences, CA). TNF-α and IL-6 levels in the serum were measured by ELISA in accordance with respective manufacturers' instructions.

### Real-Time Quantitative Reverse Transcriptase PCR

Lungs were harvested at 20 hours after CLP and stored at −80° C. Lung tissue was crushed and approximately 100 mg of tissue powder was homogenized using sonication, 3-mercaptoethanol, and the provided lysis buffer from Illustra RNAspin Mini RNA Isolation kit (GE Healthcare, Chicago, IL). Total tissue RNA was extracted according to the manufacturer's instructions. RNA was reverse-transcribed into complementary deoxyribonucleic acid (cDNA) using murine leukemia virus reverse transcriptase (Applied Biosystems; Thermo Fisher Scientific, Waltham, MA). PCR reaction was performed in a final volume of 21 μL containing 2 μg cDNA, 0.1 μmol of forward and reverse primers, 10 μL SYBRGreen PCR Master Mix (Applied Biosystems), and 7.75 μL nuclease-free water. Amplification and analyses were conducted in a Step One Plus real-time PCR machine (Applied Biosystems, Thermo Fisher Scientific). Mouse 3-actin mRNA served as an internal control for amplification. Relative gene expression was calculated using ΔΔCT methods. Relative expression of mRNA was determined as fold change relative to sham tissues.

### Lung Myeloperoxidase (MPO)

A total of approximately 50-100 mg of liquid nitrogen-based powdered lung tissues were homogenized in KPO4 buffer containing 0.5% hexa-decyl-trimethyl-ammonium bromide (Sigma-Aldrich, St. Louis, MO) using a sonicator with the samples placed on ice. Samples were subjected to two freeze/thaw cycles and centrifuged (12,000 g for 15 minutes). The supernatant was then collected and diluted in a reaction solution containing O-Dianisidine dihydrochloride (Sigma-Aldrich) and H2O2 (Thermo-Fisher Scientific, Waltham, MA) as a substrate. MPO activity was calculated by determining the rate of change in optical density (ΔOD) between 1 and 4 minutes measured at 460 nm.

### Organ Injury

Serum levels of aspartate transaminase (AST), alanine transaminase (ALT), and lactate dehydrogenase (LDH) were determined using calorimetric enzymatic assays (Pointe Scientific, Canton, MI) according to the manufacturer's instructions.

### Lung Histopathology

At time of collection, lungs were fixed in 10% formalin. At the time of processing, lungs were embedded in paraffin, cut into 5-um sections, and then stained with H&E. To evaluate for degree of lung injury, sections were analyzed under light microscopy and scored according to the system created by American Thoracic Society as previously described (32). Briefly, lung images were blindly scored from 0 to 1 based on the presence of proteinaceous debris in the airspaces, degree of septal thickening, and neutrophil infiltration in the alveolar and interstitial spaces. Lung fields at ×200 magnification were scored and averaged for analyses.

Terminal Deoxynucleotidyl Transferase dUTP Nick End Labeling (TUNEL) Assay

TUNEL staining was performed on 5-μm lung sections using a commercially available fluorescence In Situ Cell Death Detection Kit (Roche Diagnostics, Indianapolis, Ind), according to the manufacturer's instructions. 4′,6-diamidino-2-phenylindole (DAPI, Vectashield AntifadeMounting Media, H-2000) was used as a nuclear counterstain. Images were analyzed for TUNEL (+) cells by using Image J, FIJI software. (31) Microscope fluorescent intensity and exposure settings as well as Image J positivity thresholds were kept standard to reduce heterogenicity and bias in analyses.

### Protein-Protein Interaction Computational Modeling

The amino acid sequence of mouse CIRP (P60824) was retrieved from the Uniprot database. The model was generated using Iterative Threading ASSEmbly Refinement (I-TASSER) server based on templates identified by threading approach to maximize the percentage identity, sequence coverage and confidence (33). The CIRP structure has RNA binding domain (aa 6-84), disordered region (aa 70-172) and polar residues (aa 143-172). The model was refined based on repetitive relaxations by short molecular dynamics simulations for mild (0.6 ps) and aggressive (0.8 ps) relaxations with 4 fs time step after structure perturbations. The model refinement enhanced certain parameters including Rama favored residues and decrease in poor rotamers. The peptide sequences were derived by peptide selection approach and then the tertiary structures were generated using APPTEST tool which is a protocol that employs neural network architecture and simulated annealing methods for the prediction of peptide tertiary structure.(34) The tertiary peptide models were then docked into the CIRP structure using GalaxyPepDock tool, which performs similarity-based docking by finding templates from experimentally determined structures and builds complexes using energy-based optimization allowing structural flexibility (35). The protein-peptide interaction was then analyzed using PDBePISA tool.(36) Finally, the surface area of interaction interface and thermodynamic parameters were calculated and the complex structure was visualized using PyMOL.(37).

### Statistics

Data represented in the figures are expressed as mean±SEM. One-way ANOVA was used for normally distributed data for comparison among multiple groups and Brown-Forsythe and Welch ANOVA tests were used where appropriate. Survival rates were analyzed by the Kaplan-Meier estimator and compared using a log-rank test. Significant differences between groups were determined by Tukey's method. Significance was considered for p≤0.05 between study groups. Data analyses were carried out using GraphPad Prism graphing and statistical software (GraphPad Software, version 9.2).

### Study Approval

All mouse experiments were performed in accordance with and with approval of the IACUC (protocol 2021-031) at Feinstein Institutes for Medical Research (Manhasset, NY).

### Results

MFG-E8 is a Novel Scavenging Molecule of eCIRP and Facilitates its Clearance by αᵥβ₃-Integrin

To investigate the binding potential between eCIRP and MFG-E8, we utilized a computational model to predict possible interactions between murine CIRP (NP_001366351.1) and MFG-E8 (NP_032620.2). This revealed significant surface area of interaction and a strong potential binding energy (−1.1 kcal/mol) exists between eCIRP and MFG-E8. To quantify this interaction, we performed a Biacore assay and revealed strong binding between recombinant mouse (rm) CIRP and rmMFG-E8 with an equilibrium dissociation K_{D} of 2×10⁻⁷ M. Likewise, in the context of recombinant human (rh) proteins, our Biacore data further demonstrated strong binding between rhCIRP and rhMFG-E8 with a K_{D} of 2.9×10⁻⁸ M.

To investigate the sequelae of eCIRP and MFG-E8's interaction, we then focused on the potential for MFG-E8 to link eCIRP to professional phagocytes for clearance. In light of MFG-E8's role in opsonic clearance of apoptotic cells mediated through dxB3-integrin receptor-mediated endocytosis (12), we tested whether MFG-E8 could similarly enhance eCIRP cellular uptake in an avB3-integrin-dependent fashion using FITC-labeled recombinant proteins. When murine macrophage-like RAW264.7 cells were treated with FITC-eCIRP in the presence of MFG-E8, a marked increase of FITC-eCIRP uptake was visualized under confocal fluorescence microscopy compared to cells treated with FITC-eCIRP alone. Furthermore, when the αᵥβ₃-integrin receptor was blocked by a neutralizing antibody, the amount of FITC-eCIRP visualized intracellularly in the presence of MFG-E8 was remarkably reduced compared to the IgG isotype control. The quantification of these observations of fluorescence imaging, accomplished through Image J Fiji analyses, corroborated greater absolute number and area occupied by intracellular FITC-eCIRP mediated by the presence of MFG-E8, and the prevention of this internalization through blocking the αᵥβ₃-integrin receptor. Using flow cytometry, we further confirmed the enhanced internalization of FITC-eCIRP by macrophages in the presence of MFG-E8, and confirmed eCIRP's engulfment was mediated through the αᵥβ₃-integrin receptor. Together, these data indicate a strong interaction between eCIRP and MFG-E8 and demonstrate that MFG-E8 promotes the engulfment of eCIRP in macrophages through αᵥβ₃-integrin.

MFG-E8-eCIRP Complex Traffics to the Lysosomes for Degradation and Attenuates eCIRP-Induced Inflammation

To evaluate MFG-E8-mediated trafficking of eCIRP from the extracellular space to the intracellular compartment, we utilized fluorophore labeled proteins, FITC-eCIRP and AlexaFluor568-MFG-E8 and an antibody to stain for lysosomal-associated membrane protein 1 (LAMP1) to visualize lysosomes. We observed colocalization of eCIRP (green) and MFG-E8 (red) in merged images on both the cell surface and within the cell. Importantly, with the addition of LAMP1 visualization, we were able to identify colocalization of eCIRP (green), MFG-E8 (red) and lysosomes (magenta) intracellularly, suggesting the trafficking of bound eCIRP-MFG-E8 protein complexes to lysosomes to ultimately be degraded. Furthermore, to better capture eCIRP internalization, we imaged live cells with the addition of a lysosomal tracker (pink). Immediately after treatment, live RAW264.7 cells were analyzed under CO₂ insufflation on confocal fluorescent microscopy, and protein complex trafficking was captured in real time. Representative channel overlay z-stack images taken after approximately 1 hour demonstrate the intracellular colocalization of eCIRP-MFG-E8 complexes within lysosomes. These data suggest that upon extracellular binding and uptake into macrophages, eCIRP-MFG-E8 complexes are trafficked to the lysosomes where they may ultimately be degraded.

To investigate whether MFG-E8 treatment reduces eCIRP-induced inflammation, we cultured RAW264.7 cells with either PBS, MFG-E8 alone, eCIRP alone, or eCIRP co-incubated with MFG-E8 at increasing molar-equivalents of MFG-E8. We found that eCIRP significantly increased pro-inflammatory cytokines IL-6 and TNFα in cell supernatants, and the presence of MFG-E8 attenuated these levels in a dose-dependent manner. To further corroborate this effect, we cultured murine peritoneal cavity (PerC) macrophages from both wild-type (WT) and MFG-E8^{-/-} mice. In WT PerC macrophages, we again demonstrated significantly increased cell supernatant levels of IL-6 and TNFα with eCIRP treatment, and reduced levels of IL-6 and TNFα with MFG-E8 treatment. On the other hand, PerC macrophages lacking MFG-E8 protein stimulated with eCIRP exhibited significantly higher levels of IL-6 and TNFα in cell supernatants compared to WT PerC macrophages. These data suggest that MFG-E8 is a significant attenuator of eCIRP-induced cytokine release in macrophages.

Next, we evaluated the effects of these protein-protein interactions (PPIs) in vivo. We induced a sterile, pro-inflammatory state in WT and MFG-E8^{-/-} mice by injecting eCIRP intravenously. Additionally, we treated a group of WT mice with an equal amount of eCIRP however in the presence of exogenous MFG-E8. After 4 h of treatment, we observed a significant pro-inflammatory impact of eCIRP as deduced by significantly higher systemic levels of IL-6 and TNFα in the serum, as well as greater tissue injury as measured by LDH levels, compared to vehicle-treated mice. Importantly, we again observed an anti-inflammatory impact of MFG-E8, as mice treated with both eCIRP and MFG-E8 proteins had significantly reduced IL-6 and TNFα levels, as well as LDH, compared to eCIRP treatment alone. Finally, 4 hours after intravenous administration of eCIRP, MFG-E8^{-/-} mice demonstrated the highest levels of IL-6, TNFα, and LDH. In aggregate, these data demonstrate that in an eCIRP-induced pro-inflammatory state, MFG-E8 clears eCIRP to lysosomes and thereby attenuates pro-inflammatory cytokine release in vitro and in vivo.

MFG-E8 Deficiency Leads to Increased eCIRP and Inflammation in Sepsis

To better translate the importance of MFG-E8 to attenuate eCIRP-induced inflammation, we investigated these proteins in a nonsterile pro-inflammatory model, using cecal ligation and puncture (CLP) as a preclinical model of sepsis. We revealed the interplay of MFG-E8 and eCIRP in sepsis. At 20 hours after CLP-induction, although WT mice exhibited higher systemic eCIRP than sham mice counterparts, mice lacking MFG-E8 protein had significantly higher levels of systemic eCIRP than both WT CLP mice and sham mice counterparts. These data emphasize the importance of the presence of MFG-E8 to attenuate levels of eCIRP in CLP-induced sepsis. Furthermore, parallel trends were observed in pro-inflammatory cytokines and tissue injury. Specifically, serum levels of IL-6 and TNFα, as well as levels of LDH, were significantly increased in CLP WT mice compared to sham mice, and CLP MFG-E8^{-/-} mice exhibited the highest levels of IL-6, TNFα, and LDH.

Given the prevalence of secondary organ injury in severe sepsis, we investigated the impact of MFG-E8 deficiency in worsening lung injury. Utilizing the same model of CLP-induced sepsis, we collected and analyzed lung tissue for evidence of inflammation. First, we demonstrated higher lung tissue mRNA levels of IL-6, TNFα, and IL-1β in CLP WT mice compared to sham and observed the highest lung tissue mRNA levels of IL-6, TNFα, and IL-1β in CLP MFG-E8^{-/-} mice compared to CLP WT counterparts and sham. Likewise, we demonstrated higher lung myeloperoxidase (MPO) activity in CLP WT mice compared to sham and observed the highest levels of MPO activity in CLP MFG-E8^{-/-} mice compared to CLP WT counterparts and sham. In sectioned lung tissue, we found that CLP MFG-E8^{-/-} mice had significantly greater lung tissue destruction on histopathology, whereas the lung injury sustained by CLP WT mice was not as severe. Finally, in parallel with architectural distortion, we also detected a significantly higher degree of cell death in CLP MFG-E8^{-/-} mice lung tissue than CLP WT lung counterparts. In aggregate, these data highlight that MFG-E8 deficiency leads to greater levels of eCIRP, increased systemic inflammation and markers of tissue injury, and ALI in a preclinical model of sepsis.

Identification of an eCIRP-Scavenging Chimeric Peptide, MOP3

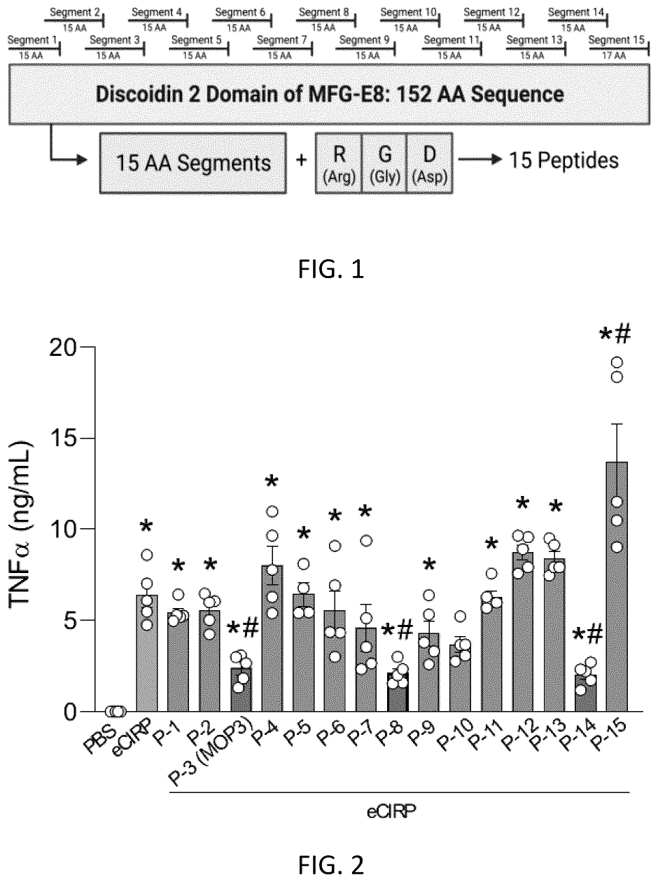

Understanding that the complex tertiary structure of the large glycoprotein, MFG-E8, may prohibit its practical use as a therapeutic, we next embarked on a strategy to develop a chimeric peptide based on the observed beneficial PPI of MFG-E8 to clear eCIRP. To start, we focused our attention on DD-2 at MFG-E8's C-terminal, given the highest potential interaction with eCIRP to occur there compared to other domains of MFG-E8. Furthermore, given that peptides of approximately 15-20 amino acids (AA) in length are ideal for maximizing specificity, stability, binding affinity and activity (22), the entire known sequence of murine MFG-E8 DD-2 (152 AA) was sectioned into 15 screening segments of approximately 15-AA's long with a 5 AA overlap. These 15 segments were then tagged with 3 additional AA at the N-terminal, Arg-Gly-Asp (RGD), given that these 3 AA (present at the N-terminal on the native MFG-E8 protein) are recognized by the integrin receptor to initiate efferocytosis. As demonstrated in FIG. 1, this process yielded 15 potential peptides of interest.

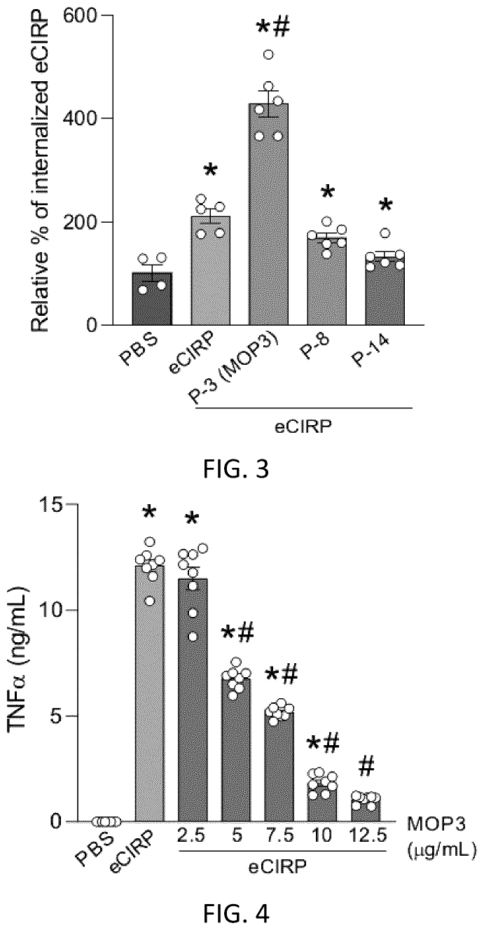

To determine if any of these 15 peptides might confer an anti-inflammatory impact, all 15 peptides underwent a screening process whereby RAW264.7 cells were stimulated with either eCIRP or eCIRP in the presence of each respective peptide. After 4 hours of treatment, cell supernatants were analyzed for TNFα. Three peptides, peptide-3, -8, and -14 were all capable of attenuating TNFα release in vitro (FIG. 2). Computational modeling was then performed on these three peptides to predict potential binding energies with eCIRP. Since our intent was to develop a peptide capable of facilitating eCIRP uptake intracellularly, we performed an additional screening experiment utilizing flow cytometry. By taking advantage of fluorophore labeled protein, FITC-eCIRP, we stimulated RAW264.7 cells with either FITC-eCIRP alone or in the presence of each peptide (peptide-3, -8, and -14). Remarkably, we discovered that only a single peptide screened, peptide-3, could increase intracellular uptake of FITC-eCIRP in macrophages (FIG. 3). Given the promising findings of peptide-3, this sequence was subsequently chosen, named “MFG-E8-derived oligopeptide 3” or “MOP3,” and all remaining experiments focused on further uncovering MOP3's mechanism of action and therapeutic potential.

Finally, to further support the mechanism of MOP3 to link eCIRP to the integrin receptor, we turned to Biacore experiments. MOP3 exhibits strong binding to eCIRP (K_{D}) of 3.1×10⁻⁷ M), and MOP3 exhibits strong binding to αᵥβ₃-integrin receptor (K_{D} of 7.76×10⁻⁷ M). Conversely, an alternative 15AA sequence from DD-2 tagged with RGD did not exhibit binding with rmCIRP, further supporting the specificity of MOP3 to bind eCIRP. Taken together, these data display the development of an effective scavenging therapeutic peptide, MOP3, and demonstrate its capability of binding eCIRP and facilitating eCIRP uptake through the integrin receptor on macrophages.

eCIRP-Induced Inflammation is Attenuated by MOP3

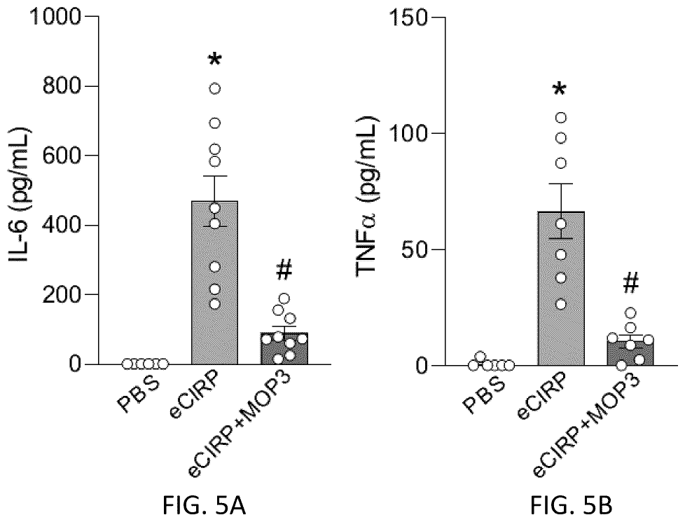

We revealed a strong inhibitory effect of MOP3 to protect against eCIRP induced TNFα levels in a dose-dependent manner in cultured RAW264.7 cells (FIG. 4). Further, we mechanistically demonstrated that macrophages stimulated with FITC-eCIRP, upon treatment with MOP3, had increased intracellular fluorescence, reflective of internalized FITC-eCIRP. When macrophages were pretreated with an antibody blocking αᵥβ₃-integrin, MOP3-mediated internalization of FITC-eCIRP was prevented. To further corroborate MOP3's ability to internalize FITC-eCIRP through RGD, a modified peptide was created whereby the Arg-Gly-Asp AAs of MOP3 were edited to Arg-Gly-Thr (RGT). MOP3-modification to “RGT” demonstrated loss of ability to internalize FITC-eCIRP in a parallel experiment. Moreover, studying this impact in vivo using a sterile, inflammatory model of eCIRP injection in WT mice, we demonstrated that intravenous MOP3 similarly protected against eCIRP-induced systemic inflammation and markers of injury, as assessed by reduced systemic levels of IL-6 and TNFα (FIG. 5A-5B). Thus, these data reveal that MOP3 effectively attenuates eCIRP-induced inflammation release in vitro and in vivo.

### MOP3 Ameliorates Inflammation, Lung Injury, and Improves Survival in Lethal Sepsis

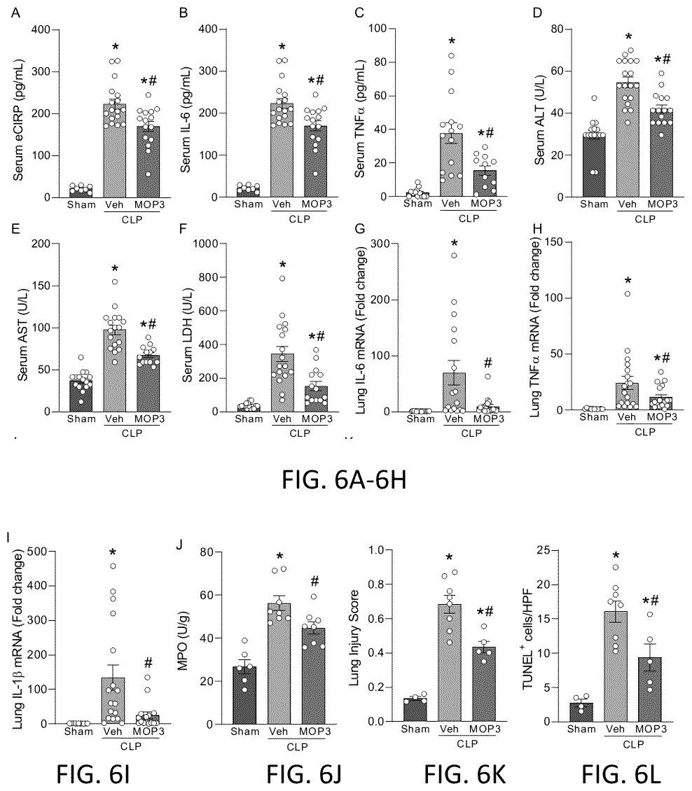

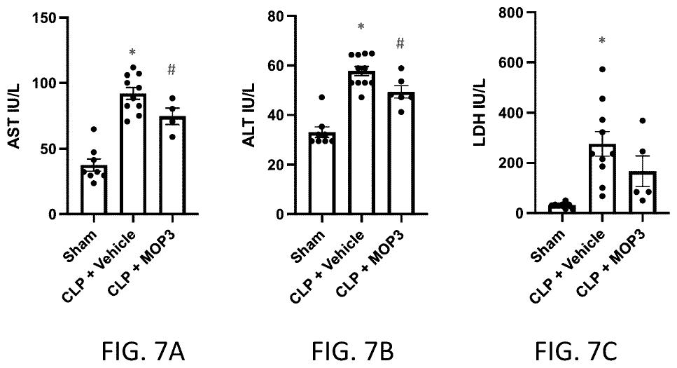

We induced sepsis in WT mice through CLP and treated mice with a one-time intravenous dose of either vehicle or MOP3 at the time of surgical closure. In this model, we found that CLP MOP3-treated mice had significantly reduced serum levels of eCIRP compared to CLP WT mice (FIG. 6A). MOP3-mediated reduction of eCIRP correlated with attenuated systemic inflammation in CLP-induced sepsis, as measured by systemic cytokines IL-6 and TNFα at 20 hours (FIG. 6B, C). Furthermore, as shown in FIG. 6D-F, we found that MOP3 treatment reduced tissue injury, as demonstrated by reduced LDH, ALT, and AST levels compared to vehicle. Together, these data support that MOP3 treatment reduces early systemic levels of eCIRP, attenuates pro-inflammatory cytokines, and decreases tissue injury (FIG. 7A-7C) in a CLP-based model of experimental sepsis.

We next sought to determine whether our chimeric therapeutic peptide, MOP3, could also protect against sepsis-induced ALI. Following the ability of MOP3 to attenuate systemic inflammation in our CLP-induced sepsis model, we found that MOP3 treatment reduced lung tissue mRNA levels of IL-6, TNFα, and IL-1ß in CLP-sepsis compared to vehicle (FIG. 6G-I). Furthermore, CLP mice treated with MOP3 exhibited decreased lung MPO levels compared to vehicle-treated mice (FIG. 6J). Finally, we analyzed sectioned lung tissue from these experiments. We observed significantly preserved histologic architecture in lungs from MOP3-treated CLP mice compared to vehicle (FIG. 6K) and significantly less apoptotic cells in lungs from MOP3-treated CLP mice compared to vehicle (FIG. 6L). These data support MOP3 to be an effective therapy in protecting against ALI in experimental sepsis. We further demonstrated that MOP3 treatment significantly reduced mortality to 17.6%, compared to 52.9% mortality of CLP vehicle-treated mice counterparts (FIG. 6M). In aggregate, these data raise the strong possibility that MOP3 may effectively reduce eCIRP and systemic inflammation, protect against inflammation, and confer improved survival in sepsis.

### Discussion

In the present study, we have identified a novel role of MFG-E8 for the opsonic clearance of eCIRP through αᵥβ₃-integrin receptor-dependent internalization and subsequent lysosome-dependent degradation of MFG-E8/eCIRP complexes to reduce inflammation. Moreover, we have developed a small therapeutic peptide that mimicked MFG-E8's function, thereby sparing the need for preparing the large parent recombinant MFG-E8 with several glycosylation sites for therapeutic applications. Together, our findings have uncovered a new function of the phagocytic protein, MFG-E8, and discovered its derivative engineered therapeutic peptide, MOP3, to clear eCIRP and reduce inflammatory sequelae in sepsis.

eCIRP inhibition has demonstrated promise in reducing inflammation and improving outcomes in sepsis. The therapeutic translation for reducing circulating levels of eCIRP is grounded in human condition, as elevated plasma levels of eCIRP have been independently correlated with a poor prognosis in patients with sepsis (4, 8). Studies with CIRP^{-/-} mice revealed protection against inflammation, further implicating eCIRP as a novel drug target for improving outcomes in sepsis and other diseases (7). Targeted eCIRP-neutralizing antibodies have previously been utilized to reduce pro-inflammatory cytokine levels, reduce tissue inflammation and organ injury, and improve survival in animal models (4). Another small molecule inhibitor of eCIRP, C23 peptide, was previously shown to have a therapeutic effect through reducing cytokine production, attenuating inflammation, protecting against liver, lung, kidney and gut injury, and improving survival in experimental sepsis models (9). Recently, another small 7-amino acid peptide, M3, was developed to block the interaction of eCIRP with its downstream receptor, and was similarly shown to inhibit systemic inflammation, protect against end-organ injury, and improve survival in murine models of sepsis (10). In contrast to previous eCIRP-targeted therapies, MOP3 is designed to perform a completely new function, whereby the detrimental effect of eCIRP is not only blocked, but rather that eCIRP is cleared from the extracellular space. By removing eCIRP from circulation through MOP3 binding and facilitating internalization, eCIRP can be degraded.

The potential therapeutic benefits of MFG-E8-mediated strategies to regulate inflammation and immunity have been previously investigated (11,18). The basis for MFG-E8's anti-inflammatory impact is rooted in its remarkable ability to recognize “eat me” signals (at the N-terminal) on apoptotic cells and link them to professional phagocytes (at the C-terminal) (11,12). However, investigations have revealed that MFG-E8's regulation of inflammation is not through clearance of apoptotic cells alone (11). For example, the clearing capacity of MFG-E8 has been revealed in other inflammatory contexts, such that MFG-E8 was found to bind and target collagen for cellular uptake in lungs in a preclinical model of pulmonary fibrosis (19). Interestingly, MFG-E8 deficient mice exhibited enhanced pulmonary fibrosis, however this was not attributed to defective apoptotic cell clearance after lung injury (19). Rather, MFG-E8^{-/-} mice were more susceptible to lung injury in this model due to their defect in collagen turnover (19). Further experiments supported the direct binding of MFG-E8 to collagen, and that MFG-E8 deficient macrophages exhibiting defective collagen uptake could be restored upon treatment with exogenous MFG-E8 containing at least one discoidin domain (19). Thus, as evidenced by the clearance of collagen, it is conceivable that MFG-E8 can recognize more than just PS signals on apoptotic cells at its discoidin domains. Likewise, here we demonstrate a new anti-inflammatory role of MFG-E8 independent of apoptotic cell clearance, where DD-2 binding of eCIRP facilitates its clearance and turnover.

Given the remarkable ability of the human body to remove billions of apoptotic cells daily through efferocytosis and maintain homeostasis, it is not surprising that therapeutic strategies have sought to harness this process for multifactorial benefits (20). On the phagocyte side, “outside-in” integrin-signaling has been a target of interest (21). Previously, a variant of annexin A5 with a gain of function modification through amino acids Arg-Gly-Asp, (“RGD,” designed to interact with the αᵥβ₃-integrin receptor) demonstrated the ability to enhance the engulfment of apoptotic cells in a human monocyte cell line (22); however, in this model, the observed increases in TNFα secretion associated with the annexin variant precluded its translation to an effective anti-inflammatory therapy (22). On the other hand, on the apoptotic cell side, exposed PS, commonly referred to as the “eat me” signal, has garnered attention as a potential target (20). For example, small molecules targeting receptors for PS are under clinical trials for inflammatory conditions and numerous cancers (20).

Since MFG-E8 is a complex glycoprotein (450 amino acids, 64-kDa) having several isoforms, preparation of recombinant MFG-E8 by recombinant DNA technology (whereby its glycosylation status is retained to preserve its optimum efficacy) is challenging (11). Therefore, a chimeric, MFG-E8-based peptide, like MOP3 (18 amino acids, 2.1-kDa) may exhibit greater clinical potential as a therapeutic candidate. Peptide drug research has become a highly attractive area in pharmaceutical development, resulting in over 170 active peptides in clinical development, with many more in preclinical studies (23, 24). Peptides are a unique class of therapeutic agents, usually with molecular weights 500-5000 Da, and confer many benefits relating to drug design, development, synthesis, and clinical utilization in enhanced efficacy and safety (25). PPI-based peptides are promising, as they can be designed with incredible specificity for cell surface receptors with high affinity and trigger intracellular effects, similar to biologics, however with lower related production costs and less immunogenicity (23,26). Thus, despite the benefits afforded by exogenous MFG-E8 itself as a treatment, the challenges associated with its clinical administration, and the demonstrated ability to harness MFG-E8's function through a designer peptide, makes MOP3 a promising new treatment for inflammatory diseases.

It is important to consider to what degree impaired apoptotic cell clearance in MFG-E8-mice contributed to worse inflammatory endpoints. Given the difficulty of untangling eCIRP clearance from the impact of apoptotic cell clearance, we utilized a model of sterile, eCIRP-induced inflammation with exogenous MFG-E8 treatment to demonstrate the impact of this PPI. Further, in nonsterile inflammation, we show that eCIRP levels are dramatically increased in MFG-E8^{-/-} mice upon septic insult. Given that MOP3 was designed to specifically bind and clear eCIRP based on MFG-E8's known sequence at DD-2, MOP3's ability to attenuate inflammation provides additional support for the impact of its predecessor, MFG′E8's ability to do the same through parallel means. Together, this evidence does not exclude the role of MFG-E8 in clearing apoptotic cells, rather, mechanistic experiments support the additional role of MFG-E8 to bind and clear eCIRP in conjunction with its previously known functions. Furthermore, although MOP3 was found to be beneficial in improving inflammatory outcomes including in a CLP-induced sepsis model in mice, we did not evaluate for any off-target effects or perform pharmacokinetic or toxicity studies. Nonetheless, lack of observed phenotypic effects from MOP3 along with improved the survival benefit afforded by MOP3 treatment in septic mice is reassuring.

Given the known deleterious effects of eCIRP, and the distinguishable and accessible steps involved in MFG-E8-mediated efferocytosis, drugging this pathway is a highly attractive strategy for intervening in a multitude of inflammatory diseases, including sepsis. The ease of development of chimeric, targeted small peptides coupled to highly drug targetable molecules, such as eCIRP, make this combination an ideal avenue for personalized medicine. Our work not only identifies a completely new mechanism by which MFG-E8 may regulate inflammation, but also introduces a novel small peptide therapeutic, MOP3, which can effectively facilitate eCIRP removal for preventing harmful inflammatory sequelae.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

### CITED PUBLICATIONS

1. Singer M, et al. The third international consensus definitions for sepsis and septic shock (Sepsis-3). JAMA. 2016; 315(8):801-10.
2. Rudd K E, et al. Global, regional, and national sepsis incidence and mortality. 1990-2017: analysis for the Global Burden of Disease Study. The Lancet. 2020; 395(10219):200-11.
3. Leng Y, et al. The Supportive Role of International Government Funds on the Progress of Sepsis Research During the Past Decade (2010-2019): A Narrative Review. Inquiry. 2022; 59:19.
4. Qiang X, et al. Cold-inducible RNA-binding protein (CIRP) triggers inflammatory responses in hemorrhagic shock and sepsis. Nat Med. 2013; 19:1489-95.
5. Ward P A. An endogenous factor mediates shock-induced injury. Nat Med. 2013; 19(11):1368-9.
6. Nofi C P, Wang P, Aziz M. Chromatin-Associated Molecular Patterns (CAMPs) in sepsis. Cell Death Dis. 2022; 13(8):700.
7. Aziz M, Brenner M, Wang P. Extracellular CIRP (eCIRP) and inflammation. J Leukoc Biol. 2019; 106(1):133-46.
8. Zhou Y, et al. The cold-inducible RNA-binding protein (CIRP) level in peripheral blood predicts sepsis outcome. PloS one. 2015; 10(9):e0137721.
9. Zhang F, et al. A cold-inducible RNA-binding protein (CIRP)-derived peptide attenuates inflammation and organ injury in septic mice. Sci Rep. 2018; 8(1):3052.
10. Denning N L, et al. Extracellular CIRP as an endogenous TREM-1 ligand to fuel inflammation in sepsis. JCI Insight. 2020; 5(5):e134172.
11. Aziz M, et al. Milk fat globule-EGF factor 8 expression, function and plausible signal transduction in resolving inflammation. Apoptosis. 2011; 16(11):1077-86.
12. Hanayama R, et al. Identification of a factor that links apoptotic cells to phagocytes. Nature. 2022; 417(6885): 182-7.
13. Matsuda A, et al. Milk fat globule-EGF factor VIII in sepsis and ischemia-reperfusion injury. Mol Med. 2011; 17(1-2): 126-33.
14. Hanayama R, et al. Autoimmune disease and impaired uptake of apoptotic cells in MFG-E8-deficient mice. Science. 2004; 304(5674): 1147-50.
15. Komura H, et al. Milk fat globule epidermal growth factor-factor VIII is down-regulated in sepsis via the lipopolysaccharide-CD14 pathway. J Immunol. 2009; 182(1):581-7.
16. Cui T, et al. Milk fat globule epidermal growth factor 8 attenuates acute lung injury in mice after intestinal ischemia and reperfusion. Am J Respir Crit Care Med. 2010; 181(3):238-46.
17. Aziz M, Prince J M, Wang P. Gut microbiome and necrotizing enterocolitis: Understanding the connection to find a cure. Cell Host Microbiome. 2022; 30(5):612-16.
18. Aziz M, et al. Current trends in inflammatory and immunomodulatory mediators in sepsis. J Leukoc Biol. 2013; 93(3):329-42.
19. Atabai K, et al. Mfge8 diminishes the severity of tissue fibrosis in mice by binding and targeting collagen for uptake by macrophages. J Clin Invest. 2009; 119(12):3713-22.
20. Mehrotra P, Ravichandran K S. Drugging the efferocytosis process: concepts and opportunities. Nat Rev Drug Discov. 2022; 21(8):601-620.
21. Torres-Gomez A, Cabañas C, Lafuente E M. Phagocytic integrins: activation and signaling. Front Immunol. 2020; 11:738.
22. Schutters K, et al. Cell surface-expressed phosphatidylserine as therapeutic target to enhance phagocytosis of apoptotic cells. Cell Death Differ. 2013; 20(1):49-56.
23. Wang L, et al. Therapeutic peptides: current applications and future directions. Signal Transduct Target Ther. 2022; 7(1):48.
24. Zaykov A N, Mayer J P, DiMarchi R D. Pursuit of a perfect insulin. Nat Rev Drug Discov. 2016; 15(6):425-39.
25. Henninot A, Collins J C, Nuss J M. The current state of peptide drug discovery: back to the future? J Med Chem. 2018; 61(4): 1382-414.
26. Fosgerau K, Hoffmann T. Peptide therapeutics: current status and future directions. Drug Discov Today. 2015; 20(1):122-8.
27. Commisso C, et al. Macropinocytosis of protein is an amino acid supply route in Ras-transformed cells. Nature. 2013; 497(7451): 633-7.
28. Nofal M, et al. mTOR inhibition restores amino acid balance in cells dependent on catabolismof extracellular protein. Mol Cell. 2017; 67(6):936-46.
29. Huynh K K, et al. LAMP proteins are required for fusion of lysosomes with phagosomes. EMBO J. 2007; 26(2):313-24.
30. Pierzyńska-Mach A, Janowski P A, Dobrucki J W. Evaluation of acridine orange, LysoTracker Red, and quinacrine as fluorescent probes for long-term tracking of acidic vesicles. Cytometry A. 2014; 85(8):729-37.
31. Schindelin J, et al. Fiji: an open-source platform for biological-image analysis. Nat Methods. 2012; 9(7):676-82.
32. Matute-Bello G, et al. Acute Lung Injury in Aminals Study Group, An official American Thoracic Society workshop report: features and measurements of experimental acute lung injury in animals. Am J Respir Cell Mol Biol. 2011; 44(5): 725-38.
33. Yang J, et al. The I-TASSER Suite: protein structure and function prediction. Nat Methods. 2015; 12(1):7-8.
34. Timmons P B, Hewage C M. APPTEST is a novel protocol for the automatic prediction of peptide tertiary structures. Brief Bioinform. 2021; 22(6):bbab308.
35. Lee H, Seok C. Template-Based Prediction of Protein-Peptide Interactions by Using GalaxyPepDock. Methods Mol Biol. 2017; 1561:37-47.
36. Krissinel E, Henrick K. Inference of macromolecular assemblies from crystalline state. J Mol Biol. 2007; 372(3): 774-97.
37. Sanner M F, Olson A J, Spehner J C. Reduced surface: an efficient way to compute molecular surfaces. Biopolymers. 1996; 38(3):305-20.

What is claimed is:

1. a method of treating a subject with sepsis or with an inflammatory condition comprising administering to the subject a therapeutic amount of a peptide selected from the group consisting of one or more of MOP3, MOP3H, MOP8 and MOP14, or a nucleic acid encoding the peptide, wherein
MOP3 has the amino acid sequence
(SEQ ID NO. 1)
RGDSSSYKTWNLRAFGWY,
MOP3H has the amino acid sequence
(SEQ ID NO. 16)
RGDSSSYKTWGLHLFSWN,
MOP8 has the amino acid sequence
(SEQ ID NO: 8)
RGDVTGIITQGARDFGHI,
and
MOP14 has the amino acid sequence
(SEQ ID NO: 14)
RGDPFMARYVRVLPVSWH.

2. the method of claim 1, consisting of administering one or both of MOP3 and MOP3H to the subject.

3. The method of claim 1, wherein treatment of the subject with the peptide reduces tissue injury in the subject compared to tissue injury that would occur in the absence of treatment with the peptide.

4. The method of claim 3, wherein lung injury is reduced by treatment of the subject with the peptide.

5. The method of claim 1, wherein the inflammatory condition is one or more of appendicitis, peptic, gastric or duodenal ulcers, peritonitis, pancreatitis, ulcerative colitis, pseudomembranous, acute or ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, necrotizing enterocolitis, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, bums, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, adult respiratory distress syndrome, Alzheimer's disease, meningitis, encephalitis, multiple sclerosis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcet's syndrome, allograft rejection, graft-versus-host disease, ankylosing spondylitis, Type I diabetes, ankylosing spondylitis, Berger's disease, reactive arthritis (Reiter's syndrome) or Hodgkin's disease.

6. The method of claim 1, wherein the inflammatory condition is one or more of appendicitis, peptic, gastric or duodenal ulcers, peritonitis, pancreatitis, ulcerative colitis, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, necrotizing enterocolitis, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection, graft-versus-host disease and bacterial infection.

7. A pharmaceutical composition comprising a pharmaceutically acceptable carrier and a peptide selected from the group consisting of one or more of MOP1, MOP2, MOP3, MOP3H, MOP4, MOP5, MOP6, MOP7, MOP8, MOP9, MOP10, MOP11, MOP12, MOP13, MOP14 and MOP15.

8. The pharmaceutical composition of claim 7, wherein the peptide is one or both of MOP3 and MOP3H.
